(19) United States
(12) Patent Application Publication
(10) Pub. No.: US 2014/0323937 A1
(43) Pub. Date: Oct. 30, 2014

(54) KNEE BRACE WITH ADJUSTABLE BOLSTER

(71) Applicant: Steven S. Knecht, Bakersfield (CA)

(72) Inventor: Steven S. Knecht, Bakersfield (CA)

(73) Assignee: Townsend Industries, Inc. d/b/a Townsend Design, Bakersfield, CA (US)

(21) Appl. No.: 14/359,157

(22) PCT Filed: Sep. 14, 2012
(86) PCT No.: PCT/US2012/055449

§ 371 Date: May 19, 2014

Related U.S. Application Data
(60) Provisional application No. 61/535,397, filed on Sep. 16, 2011.

(51) Int. Cl. A61F 5/01

(52) U.S. Cl. CPC A61F 5/0125
USPC 602/16

(57) ABSTRACT

A knee brace which allows the same bolster pad to be used for either the right or left leg, is adjustable to compensate for the differing angle between men and women and allows individual adjustment for each patient's comfort and brace rotation for proper fit and joint alignment. These characteristics are obtained by a contoured bolster that is selectively mountable at various positions on either side of the shin cross piece depending on whether the brace is for the left or right leg, the bolster always being on the medial side of the shin to contact the flat section on the front of the shin thereby minimizing unwanted rotation of the brace. The bolster pad has a front face with a curvature matched to a curvature of an inner side of the cross piece and a flat on a rear side to contact the front of the shin.

### CROSS REFERENCE TO RELATED APPLICATION

This application is a non-provisional conversion of provisional application 61/535,397.

### BACKGROUND OF THE INVENTION

1. Field of the Invention

The present invention relates to orthotic knee braces, and in particular, to pads and bolsters for such braces.

2. Description of Related Art

The use of pads on straps and cross members of orthopedic braces. For example, U.S. Pat. No. 6,969,364 entitled “Anatomically designed orthopedic knee brace” assigned to GENERATION II USA INC discusses an orthopedic knee brace for controlling movement of a wearer's knee. The thigh and calf engaging members are manufactured from a laminate combination including a soft compressible material, a loop material, and a hydrophilic breathable material. The soft compressible type material accommodates pressure distribution is provided in a semi-elliptical shape to accommodate different leg shapes without causing localized pressure points to the wearer's leg by the thigh engaging member and the calf engaging member made of soft compressible material. The description appears to indicate that a hook-and-loop type material is used to attach the member to the cross piece. However, no variable positioning capability is described or apparently obtainable, and no use for a calf cross piece is suggested.

In U.S. Pat. No. 7,963,933 entitled “Osteoarthritis knee orthosis” invented by Richard A. Nace, an improved osteoarthritis knee brace for treating a knee joint of a patient suffering from osteoarthritis of the knee is disclosed. The knee brace also includes a soft adjustable cushion pad on the posterior calf strap. The soft adjustable cushion pad is intended to eliminate potential pressure and shear on the patient's skin and is attached to the strap by hook and loop type fastening material. However, the pad is of a length that precludes varying of its positioning and no use for a calf cross piece is suggested.

U.S. Pat. No. 6,524,265 entitled “Leg brace support structure” invented by Theodore B. Hogg discloses an orthopedic leg brace and an improved longitudinal support structure that provides support to a wearer's leg. This patent shows a pad that is attached at one end by a screw to a cross piece and via a hook and loop type connection to a strap at its other end. One end of the cross piece has several sets of holes to enable the position of the cross piece on the vertical strut to be adjusted by selection of a particular one of the sets of holes. However, this pad is for the ankle area, has a flat contour, and is not designed for adjustable positioning.

### SUMMARY OF THE INVENTION

The present invention is designed to allow the same bolster pad to be used for either of the right and left legs, is adjustable to compensate for the differing angle between men and women and allows for individual adjustment for each patient's comfort and brace rotation for proper fit and joint alignment.

The above characteristics are obtained by a contoured bolster that is able to be selectively mounted at various positions on either side of the shin cross piece depending on whether the brace is for the left or right leg, the bolster always being on the medial side of the shin to contact the flat section on the front of the shin thereby minimizing unwanted rotation of the brace.

### BRIEF DESCRIPTION OF THE DRAWINGS

FIG. 1 is a partial, front elevational view of a knee brace in accordance with the invention with all posterior straps removed;

FIG. 2 is a front perspective view of a knee brace in accordance with the invention;

FIG. 3 is a partial, rear elevational view of a knee brace in accordance with the invention all posterior straps removed;

FIG. 4 is a partial, side elevational view of a knee brace in accordance with the invention all posterior straps removed;

FIG. 5 is a view similar to FIG. 2, but with a bolster shown just prior to mounting;

FIG. 6 is a rear perspective view of the brace and bolster shown in FIG. 5;

FIG. 7 is a top view of the brace and bolster shown in FIG. 5;

FIG. 8 is a top view of the brace with the bolster mounted;

FIG. 9 is a perspective view of the shin cross member of present invention;

FIGS. 10(a)-(d) are rear, rear perspective, top plan and side elevational views of the bolster of the present invention; and

FIG. 11(a) is a plan view of the shin cross member without a bolster attached and FIGS. 11(b)-(i) are plan views of the shin cross member with the bolster attached in various positions.

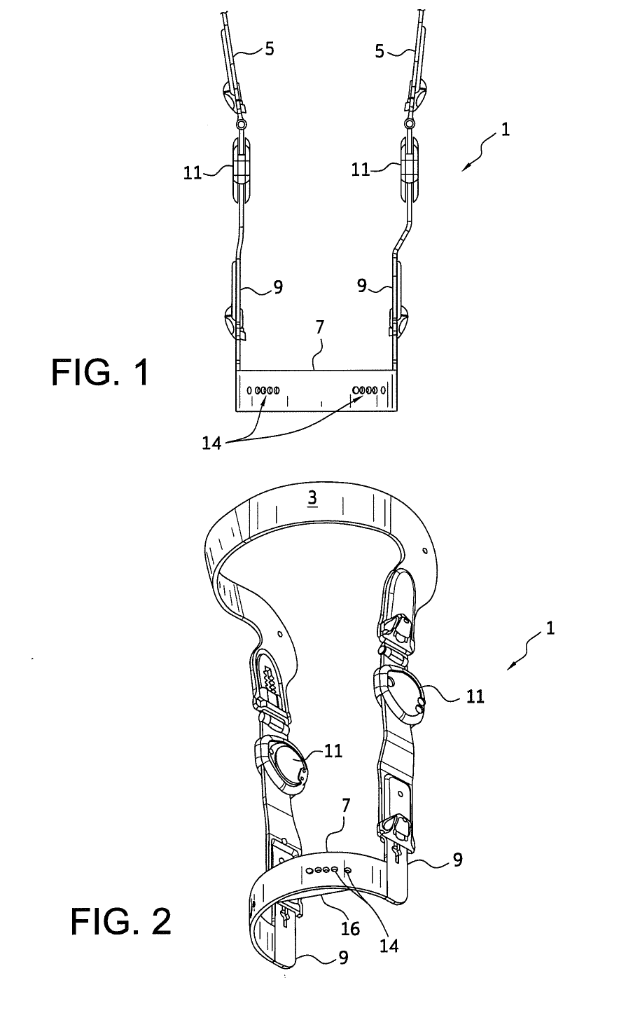

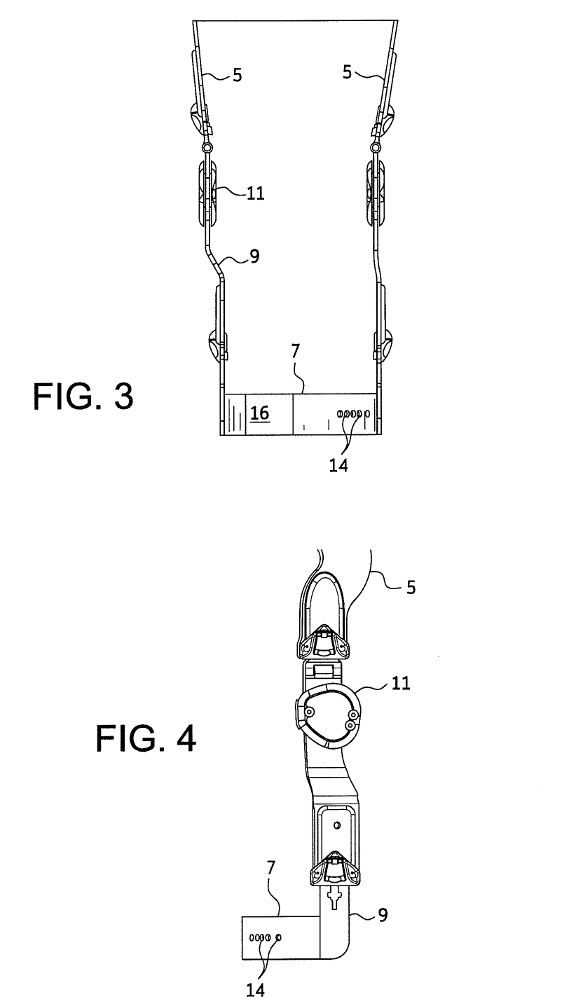

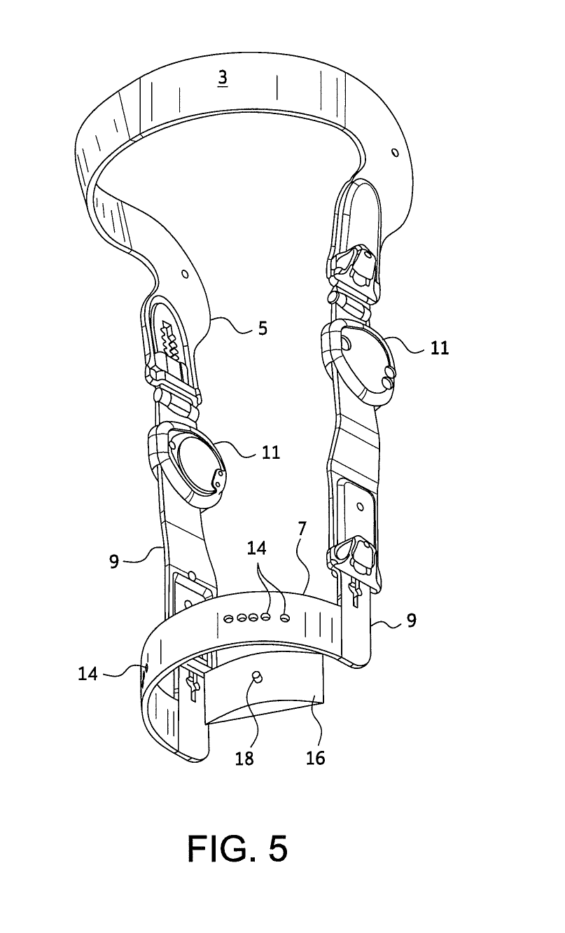

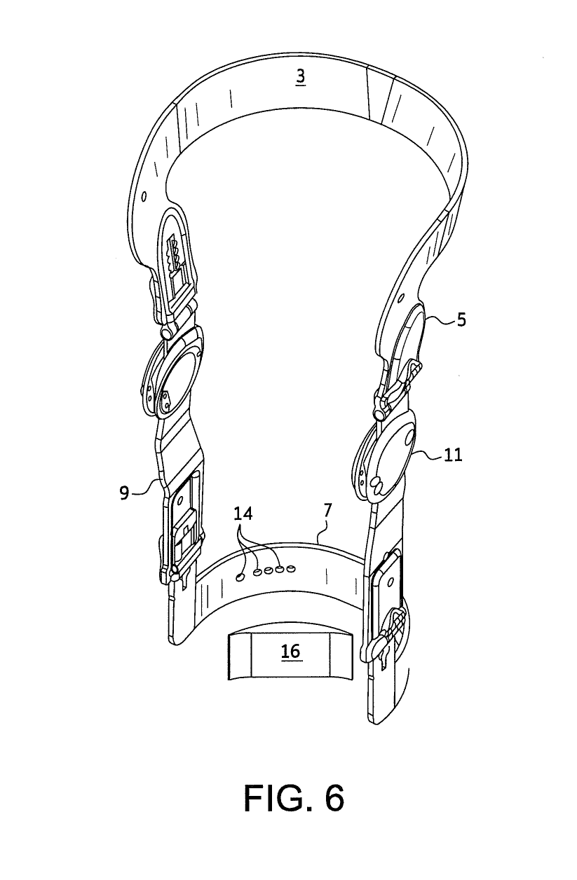

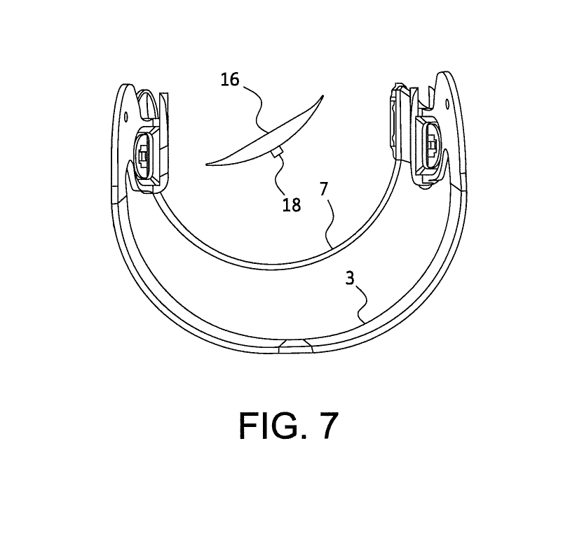

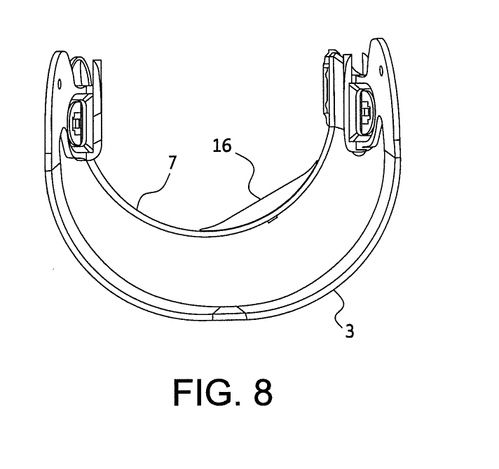

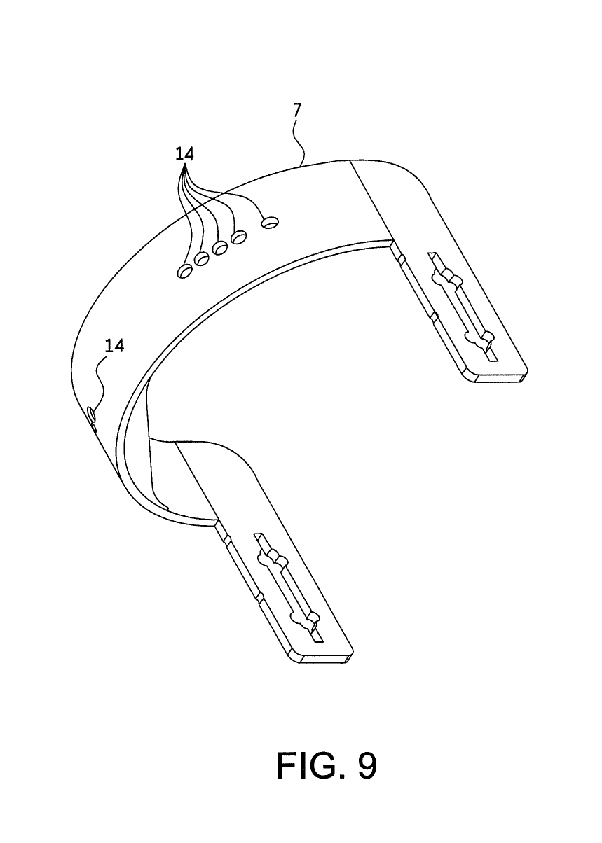

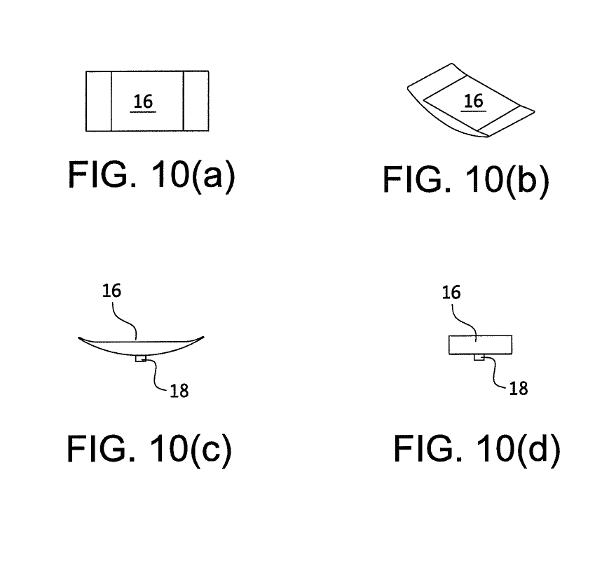

### DETAILED DESCRIPTION OF THE INVENTION

At the outset, it is noted that the orthotic knee brace shown in the drawings is merely an illustrative example and the following description of the invention should be viewed as applicable to numerous other types of knee braces, such as that of the present inventor's U.S. Pat. No. 6,981,957. As can be seen in the drawings, as is typical, the knee brace 1 has an upper cross piece 3 connecting the medial and lateral femoral arms 5, a lower cross piece 7 connecting the medial and lateral tibial arms 9, and a hinge mechanism 11 connecting the lateral femoral and tibial arms and connecting the medial femoral and tibial arms. Not shown are the upper and lower posterior straps that connect to medial and lateral sides of the cross pieces and/or medial and lateral femoral and tibial arms to hold the brace on the wearer's leg.

Where the present invention departs from typical knee braces will now be described.

As can be seen, the lower or shin cross piece 7 has two sets of holes 14, one set at each side near the corresponding lateral or medial tibial arm 9. However, the provision of holes 14 on both sides of the cross piece is to allow a single cross piece be usable for both left and right leg braces. In a given case, only the holes which are located on the medial side of the leg of the user are usable since the bolster is to contact the flat section on the front of the shin to thereby minimize unwanted rotation of the brace.

A circular sector shaped pad or bolster 16 made of a fairly dense foam material and has a mounting pin 18 projecting from the center of its front face. The pin 18 is sized to snap into any of the holes 14 and held by a friction fit in the selected one of the holes 14; although, in the case where the bolster is covered by a conventional cross piece cushion pad, the pad and leg will hold the bolster against the cross piece so that a secure direct attachment between the bolster and cross piece is not required, e.g., the pin can fit in the hole without a friction fit or a piece of double-sided adhesive tape could be used to position and hold the bolster against the cross piece. The curvature of the front face of the bolster 16 is matched to the curvature of the inner side of the cross piece 7.

The pin 18 can be secured to the bolster in various ways, such as by being attached to a plate or having radially projecting arms which are embedded in the material of the bolster. Alternatively, the pin can be part of an over-molded urethane covering of the dense foam material of the bolster.

Instead of using a friction fit pin connection, each of the sets of holes could be replaced by a slot to enable a finer degree of positional adjustment. In such a case, a fastener would be needed to fix the bolster 16 in place. For example, the pin could be made long enough to project out of the opposite side of the cross piece 7 with the projecting end being threaded to receive a wing nut or other fastener which can be tightened to hold the bolster in place.

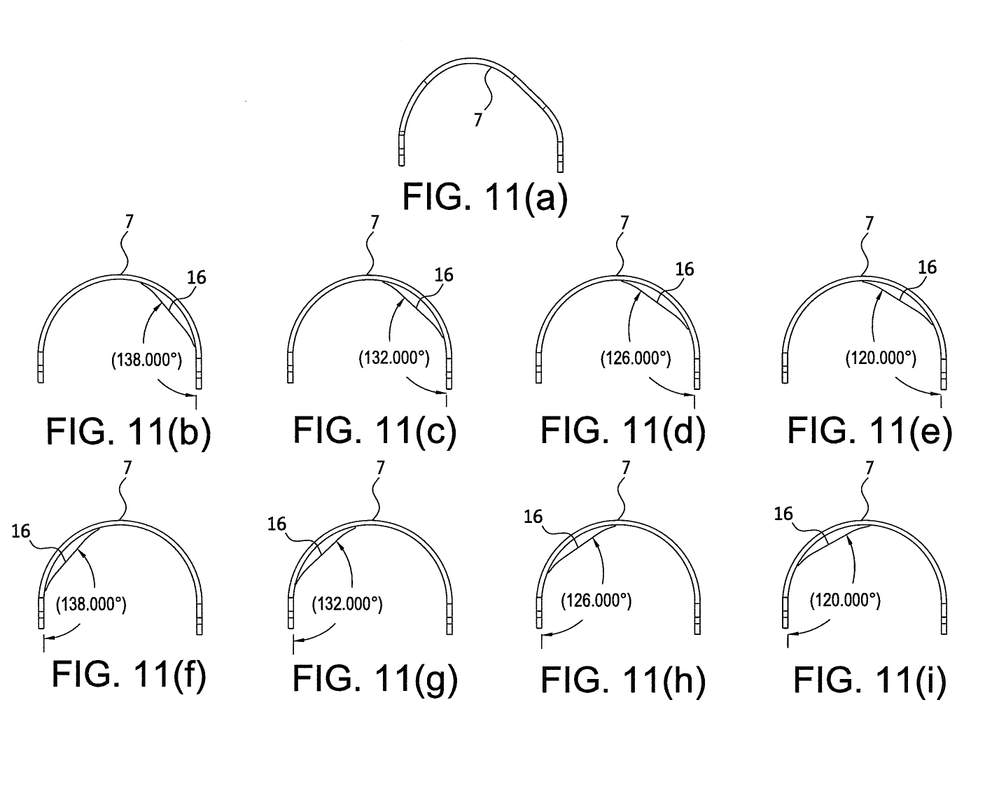

As can be seen from FIG. 11, which shows the left tibial cross piece 7, by selection of an appropriate hole 14, the bolster 16 can be mounted to the inner side of the cross piece 7 in any of various angular positions. In this regard, the positions shown are representative only and various other angular positions can be obtained. By having holes at both sides of the cross piece 7, as noted above, the same cross piece can be used for both the right and left leg knee braces; thus, FIGS. 11(b)-11(e) represent positions for the left leg while FIGS. 11(f)-11(i) represent positions for the right leg. Furthermore, the adjustability of the bolster position allows the differing angle between men and women to be accounted for and for individual adjustment for each patient's comfort as well as minimizing brace rotation for proper fit and joint alignment.

The size of the bolster 16 will be based on the size of the brace. While only a given size bolster may be appropriate for a particular brace, it is contemplated that bolsters of different densities may be provided for each brace in order to aid in providing a comfortable fit for a particular user.

1. An knee brace comprising:
a medial tibial arm,
a lateral tibial arm,
a medial femoral arm,
a lateral femoral arm,
an upper cross piece connecting the medial and lateral femoral arms,
a lower cross piece connecting the medial and lateral tibial arms
a hinge mechanism connecting the lateral femoral and tibial arms,
a hinge mechanism connecting the medial femoral and tibial arms, and
a circular sector shaped pad or bolster made of a foam material having a front face with a curvature matched to a curvature of an inner side of the lower cross piece and a leg facing side that is flat to fit against the flat section on the front of the wearer's shin so as to minimize rotation of the brace, and
an arrangement for holding the pad or bolster against of the inner side of the lower cross piece in any of various angular positions at a medial half of the lower cross piece.

2. Knee brace according to claim 1, wherein said arrangement for holding the bolster against the inner side of the lower cross piece comprises a pin on the pad or bolster which can selectively fit in any one of a sets of holes located on a medial half of the lower cross piece.

3. Knee brace according to claim 1, wherein said arrangement for holding the bolster against the inner side of the lower cross piece comprises a pin on the pad or bolster which is selectively positionable in any of various positions along a slot located extending along a medial half of the lower cross piece and a fastener for securing the pad or bolster in the position selected.

4. Knee brace according to claim 1, wherein said arrangement for holding the bolster against the inner side of the lower cross piece comprises a piece of double-sided adhesive tape.

5. Knee brace according to claim 1, wherein said pad or bolster is one of a plurality of bolsters of different densities, each of which is selectively mountable to the lower cross piece to aid in providing a comfortable fit for a particular user.
